(19) United States
(12) Patent Application Publication
(10) Pub. No.: US 2026/0240780 A1
(43) Pub. Date: Aug. 20, 2026

(54) COATING COMPOSITION AND COATED TABLET

(71) Applicant: BASF SE, Ludwigshafen am Rhein (DE)

(72) Inventors: Thorsten AGNESE, Ludwigshafen am Rhein (DE); Thorsten CECH, Ludwigshafen am Rhein (DE); Felicitas GUTH, Ludwigshafen am Rhein (DE)

(21) Appl. No.: 19/164,621

(22) PCT Filed: Mar. 14, 2024
(86) PCT No.: PCT/EP2024/056865

§ 371 Date: Sep. 12, 2025

(30) Foreign Application Priority Data
Mar. 15, 2023 (EP) 23162076.6
Jan. 19, 2024 (EP) 24152840.5

(51) Int. Cl. A61K 9/28; A61K 31/4422; A61K 47/14; A61K 47/20

(52) U.S. Cl. CPC A61K 9/284; A61K 9/2813; A61K 9/2853; A61K 9/2893; A61K 31/4422; A61K 47/14; A61K 47/20

(57) ABSTRACT

A coating composition useful for coating tablets, such as compressed tablets, caplets, pills, capsules, confectionary or seeds comprises an opacifying amount of crospovidone having an average particle size of less than 10 μm. Crospovidone having an average particle size of less than 10 μm is used as opacifier and white pigment in a coating of a coated tablet for replacement of titanium dioxide.

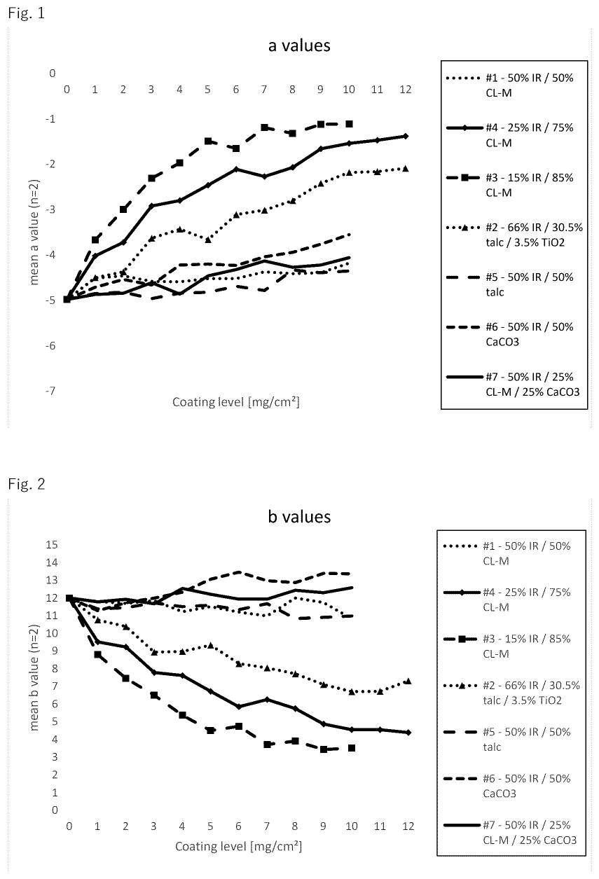

The present invention relates to a coating composition, in particular a film coating composition, a coated tablet which has the coating, and the use of crospovidone having an average particle size of less than 10 μm as opacifier and white pigment in a coating.

Film coating is a process in which the substrate, such as a tablet, a pellet, a confectionary or a seed, is enveloped by a thin layer of polymeric film forming material. Film coating may be carried out for a wide variety of reasons such as changing the color for aesthetic purposes or branding; to protect the material from degradation due to environmental conditions such as moisture, oxygen or light; to modify the release of the active ingredient of a pharmaceutical composition; to alleviate bitterness; and/or to reduce release of an odor. The film coating is generally provided on a tablet by using a film former. Film coating of pharmaceutical tablets allows efficient, controlled, uniform and reproducible coats.

Coating formulations typically contain opacifiers and white pigments to provide a dedicated color coverage. Without those components, the coat remains translucently opaque and the substrate, e.g., a tablet core, remains visible. In the pharmaceutical industry, the standard white pigment is titanium dioxide, and the standard opacifier is talc.

In an opinion letter of efsa (European Food Safety Authority), published May 2021, titanium dioxide is no longer considered safe due to uncertainty of genotoxicity due to nanoparticles contained. Subsequently, a food ban was announced in January 2022. Even though the pharmaceutical industry is currently not affected, pharmaceutical companies have started to reformulate their drug products to be prepared for a potential ban.

A huge variety of components such as, e.g., calcium carbonate, magnesium oxide, tricalcium phosphate, have been evaluated by formulators. However, besides the fact that some of these excipients contain nanoparticles as well, or are not inert and tend to react with either polymer or active pharmaceutical ingredients (API), the processing is demanding. The color coverage is poorer compared to the traditional formulations, and due to the high specific density of some of the pigments suggested for replacement of titanium dioxide, sedimentation of the pigments within the coating dispersion is strongly pronounced. Consistently, a high coating level is required, and poor process reliability is observed. To date, there is no solution available to formulators that matches the performance of titanium dioxide.

US 2012/0164223 discloses a rapidly disintegrating, solid coated dosage form comprising a solid core coated with a film coating. The film coating comprises completely or partially hydrolyzed, rapidly water-soluble polyether-vinyl ester graft polymers and may contain up to 30% by weight of a finely divided crospovidone with an average particle diameter between 2 and 30 μm.

B. Hancock et al., “Titanium Dioxide (E171 Grade) and the Search For Replacement Opacifiers and Colorants: Supplier Readiness Survey, Case Studies and Regulatory Perspective” in: Journal of Pharmaceutical Sciences, describe investigations on various TiO₂ replacements as opacifiers in solid dosage forms. The properties, e.g. regarding whiteness, realized using TiO₂ as opacifier could so far not be replicated with the described replacements.

The invention relates to a coating composition, in particular a film coating composition, comprising an opacifying amount of crospovidone having an average particle size of less than 10 μm. The coating composition is useful for coating tablets, such as compressed tablets, caplets, pills, capsules, confectionary or seeds. Tablets include pharmaceutical tablets and nutritional supplements.

“Crospovidone” is a name customary in the pharmaceutical sector for water-insolubly crosslinked polyvinylpyrrolidone. Such substances are commercially available, for example, as Kollidon® CL, from BASF SE, Germany, and also as Polyplasdone® XL from International Specialty Products, USA. These substances have hitherto been used as disintegrants, or in micronized form as suspension stabilizers.

Crospovidones useful in the present invention have an average particle size of less than 10 μm, for example less than 8 μm, e.g., 2 to 5 μm. Quite unexpectedly, it was found that these crospovidones can be used as a substitute for both talc and titanium dioxide in coatings. It has been found that the incorporation of crospovidone provides coating compositions having excellent whiteness, shielding power, and coloring power.

Crospovidones having very small particle sizes obtained by micronization are the crospovidones Kollidon® CL-M from BASF and Polyplasdone® INF-10 from International Specialty Products. Kollidon CL-M has a particle size D[4,3] of 3 to 10 μm. Crospovidone grades with an even finer particle size could be useful, e.g., for engraved tablets and could reduce the risk of bridging, particularly when higher coating levels are required. Kollidon CL-M is a pharmaceutical excipient. It is a standard excipient with regulatory freedom, pharmacopoeia monographs and precedence of use in registered drug products.

The average particle sizes is stated as volume mean diameter, also referred to as volume moment mean (De Brouckere mean diameter) D[4,3] in micrometers. D[4,3] is defined as

$$D [ 4 , 3 ] = \frac{\sum {n}_{i} {D}_{i}^{4}}{\sum {n}_{i} {D}_{i}^{3}}$$

where nᵢ is the frequency of occurrence of particles in size class i, having a mean Dᵢ diameter. The size distribution is suitably determined by means of laser defractometry using a Malvern Mastersizer X at a compressed-air atomization of 2 bar.

An opacifying amount of crospovidone having an average particle size of less than 10 μm is the amount which imparts a desired degree of opacity, preferably comparable to that obtained in conventional coatings using inorganic materials such as titanium dioxide and talc. Generally, the opacifying amount is 25 wt.-% or more, preferably 32 wt.-% or more, or 40 wt.-% or more, in some cases 50 wt.-% or more, relative to the solids content of the coating composition. Generally, the opacifying amount is 85 wt.-% or less, or 75 wt.-% or less, relative to the solids content of the coating composition. The actual opacifying amount may depend on a number of factors including the original color of the substrate, the intended color impression, and the pigment binding capacity of the film-former.

In some cases, it may be desirable to impart a specific observable color, coloration or hue to the coated tablet. To this end, the coating composition may additionally comprise a colorant and/or a pigment other than crospovidone. The combination of crospovidone with a colorant and/or a pigment has been found to provide clear and bright color tones.

The colorant and/or pigment may be any colorant and/or pigment used in making coating dispersions for pharmaceutical tablets and the like.

Herein, the abbreviation “FD&C” is short for “Food, Drug, and Cosmetic”. FD&C colors are colors approved by the United States “Federal Food, Drug, and Cosmetic Act” for food, drugs and cosmetics. Analogously, the abbreviation “D&C” is short for “Drug, and Cosmetic”.

A “lake” is an insoluble material that tints by dispersion. Lakes are produced by coloring aluminum salt substrates using FD&C dyes.

Suitably, the colorant is selected from natural and synthetic colorants. For example, the colorant is selected from water-soluble colorants such as
azo dyes;
FD&C colors such as FD&C Blue No. 1 (Brilliant blue FCF), FD&C Blue No. 2 (Indigo carmine), FD&C Green No. 3 (Fast Green FCF), FD&C Red No. 3 (Erythrosine), FD&C Red No. 4 (Disodium Salt), FD&C Red No. 40 (Allura Red AC), FD&C Yellow No. 5 (Tartrazine), and FD&C Yellow No. 6 (Sunset Yellow FCF);
D&C colors such as D&C Green No. 5 (Acid Green 25), D&C Green No. 6 (Solvent Green 3), D&C Green No. 8 (Solvent Green 7), D&C Orange No. 4 (Acid Orange 7), D&C Orange No. 5 (Acid Orange 11), D&C Red 17 (Sudan III), D&C Red 21 (Solvent Red 43), D&C Red 22 (Acid Red 87), D&C Red 27 (Solvent Red 48), D&C Red 28 (Acid Red 92), D&C Red 33 (Acid Red 33), D&C Yellow No. 8 (Acid Yellow 73), D&C Yellow No. 10 (Acid Yellow 3), D&C Yellow No. 11 (Solvent Yellow 33), and External D&C Violet No. 2 (Acid Violet 43);
and mixtures thereof.

Suitably, the pigment is selected from natural and synthetic pigments. For example, the pigment is selected from
red, yellow and black iron oxides;
calcium carbonate;
magnesium carbonate;
zinc oxide;
pyrogenic silica;
FD&C lakes such as FD&C Red No. 40 Aluminum Lake (Allura Red), FD&C Blue No. 1 Aluminum Lake (Brilliant Blue FCF), and FD&C Yellow No. 5 Aluminum Lake (Tartrazine);
D&C lakes such as D&C Red No. 6 Barium Lake (Pigment Red 57), D&C Red No. 7 Calcium Lake (Pigment Red 57:1), D&C Red No. 27 Aluminum Lake (Solvent Red 48:2), D&C Red No. 28 Aluminum Lake (Pigment Red 174), D&C Red No. 30 Talc Lake (Vat Red 1), D&C Red No. 33 Aluminum Lake (Acid Red 33), D&C Violet No. 2 (Acid Violet 43), and D&C Yellow No. 10 Aluminum Lake (Pigment Yellow 115);
mica based pearlescent pigments;
and mixtures thereof.

The coating composition may comprise the colorant and/or pigment in an amount of 0.1 to 10 wt.-%, relative to the solids content of the coating composition.

In preferred embodiments, the coating composition is free of titanium dioxide, preferably free of titanium dioxide and talc.

An advantage of crospovidone having an average particle size of less than 10 μm is its low specific density. Thus, sedimentation in the preparation vessel and tubing is low and leads to a high process reliability. Coverage and opacity of tablet surfaces are excellent and outstanding results are obtained at lower coating levels. At comparable levels crospovidone having an average particle size of less than 10 μm outperforms all alternatives. Unlike talc which is a natural product, batch to batch variability is expected to be reduced with crospovidone having an average particle size of less than 10 μm due to its synthetic nature.

The preparation of the coating formulation is simplified, as no high shear mixer for particle disaggregation is required, dispersion handling is simplified in light of the reduced sedimentation tendency, and process reliability is improved for the same reason.

The coating techniques in which the coating composition can be used are not particularly limited. They include sugar coating, organic solvent based film coating, aqueous based film coating, delayed release coating and granule coating techniques. Preferably however, the coating composition is an aqueous film coating composition. The coating composition may contain a film-former, generally a polymeric film-former. The polymeric film-former is not subject to particular restrictions and may be selected from polyvinylpyrrolidone (PVP), polyvinylpyrrolidone-vinyl acetate copolymer (co povidone), methylcellulose (MC), ethyl cellulose (EC), hydroxypropyl cellulose (HPC), hydroxypropyl methylcellulose (HPMC), sodium carboxymethyl cellulose (sodium CMC), polydextrose, starch, modified starch, polyvinyl alcohol (PVA), copolymers based on polyvinyl alcohol, polyvinyl acetate phthalate, or combinations thereof.

Preferred copolymers based on polyvinyl alcohol include acetate-polyalkylene ether graft copolymer completely or partially hydrolyzed after the polymerization. A particularly preferred polymer is Kollicoat® IR, a polyvinyl alcohol-polyether graft copolymer from BASF SE, Ludwigshafen, which corresponds to the monograph “Macrogol Poly(vinyl alcohol) Grafted Copolymer” in European Pharmacopeia 6.7. This is a polyvinyl acetate-polyethylene glycol graft copolymer with vinyl acetate units hydrolyzed after the polymerization and having a degree of hydrolysis of from 90 to 99 mol-%, stated in the European Pharmacopeia as “Polymer with 75% polyvinyl alcohol and 25% polyethylene glycol in powder form”. The viscosity as 20% strength aqueous solution is between 50 and 250 mPas. The molecular weight is between 30 000 and 150 000 daltons, the average molecular weight is preferably 45 000 g/mol.

Another preferred copolymer based on polyvinyl alcohol is Kollicoat® Protect from BASF SE, Ludwigshafen, which is a mixture of Kollicoat® IR (a polyvinyl alcohol (PVA)-polyethylene glycol (PEG) graft copolymer) and polyvinyl alcohol (PVA). Kollicoat® Protect and Kollicoat® IR are preferred polymers due to their high pigment loading capacity.

The coating composition may contain polymers for modified drug release. Such polymers include cellulose acetate phthalate, cellulose acetate trimellitate, hydroxypropyl methylcellulose phthalate, methacrylate-based polymers and the like.

Suitable methacrylate-based polymers include poly(butyl methacrylate, (2-dimethylaminoethyl) methacrylate, methyl methacrylate)) 1:2:1, poly(ethyl acrylate, methyl methacrylate) 2:1, poly(methacrylic acid, methyl methacrylate) 1:1, poly(methacrylic acid, ethyl acrylate) 1:1, poly(methacrylic acid, methyl methacrylate) 1:2, poly(methyl acrylate, methyl methacrylate, methacrylic acid) 7:3:1, poly(ethyl acrylate, methyl methacrylate, trimethylammonioethyl methacrylate chloride) 1:2:0.2, poly(ethyl acrylate, methyl methacrylate, trimethylammonioethyl methacrylate chloride) 1:2:0.1, poly(methyl methacrylate-diethylaminoethyl methacrylate) 6:4.

The amount of film-former used in the composition may be in the range of 20 wt.-% to 75 wt.-%, more preferably 24 wt.-% to 42 wt.-%.

The coating composition may contain a wetting agent, a waxy component, a plasticizer, a glidant, a flavoring agent, a sweetening agent, an additional polymer and/or other pharmaceutically acceptable excipients.

The composition of the present invention may include a plasticizer. The term “plasticizer” as used herein refers to compounds having plasticizing properties which are useful to form an integer film. The plasticizer may be used alone or in combination with other plasticizer(s). The plasticizer may be selected from phthalate esters; phosphate esters; other esters like citrates, stearates, sebacates, oleates, and adipates; oils, glycerol, and glycols. Preferably, the plasticizer is selected from ethyl phthalate, methyl phthalate, dipropyl phthalate, diethyl phthalate, glycerine, polyethylene glycol (PEG), triethyl citrate (TEC), dibutyl sebacate (DBS), diethyl phthalate (DEP), dibutyl phthalate (DBP), triacetin. More preferably, the plasticizer is selected from diethyl phthalate, triethyl citrate, dibutyl phthalate, and combinations thereof.

The amount of plasticizer used in the composition may be in the range of 5 wt.-% to 20 wt.-%, more preferably in the range of 15 wt.-% to 7 wt.-%.

The composition of the present invention may include a waxy component or a combination of waxy components. The term “waxy” as used herein refers to the substance having hydrophobic activity. The waxy component may be selected from fatty acids, glyceryl mono-stearate, glyceryl behenate, glyceryl palmitostearate, magnesium stearate, sorbitan esters, stearic acid, palmitic acid, polyoxyethylene alkyl ethers, lauroyl polyoxyl glycerides and stearoyl polyoxyl glycerides, cetostearyl alcohol, cetyl alcohol, cerosin, propylene glycol monostearate, sorbitan tristearate, sodium stearyl fumarate, stearyl alcohol, hydrogenated vegetable oil, carnauba wax, microcrystalline wax, dioctyl sodium sulfosuccinate, ethylene glycol stearates, glyceryl monooleate, lanolin, myristic acid, petrolatum/lanolin alcohols, and derivatives of stearate. The amount of waxy component used in the composition may be in the range of 0.1 wt.-% to 10 wt.-%, more preferably in the range of 1 wt.-% to 7 wt.-%.

The wetting agent used in the composition of the present invention may be selected from ammonium lauryl sulfate, sodium lauryl sulfate, polysorbate-20, polysorbate-40, polysorbate-60, polysorbate-80, more preferably polysorbate-80.

The amount of wetting agent used in the composition may be in the range of 0.1 wt.-% to 4 wt.-%, more preferably 0.5 wt.-% to 3 wt.-%.

The composition of the present invention may include a glidant. The term “glidant” as used herein refers to compounds which increase the flow of dry powders. The glidant may be selected from magnesium stearate, Aerosil® (colloidal silicon dioxide), starch, hydrophobic colloidal silica, magnesium oxide, magnesium silicate, and magnesium trisilicate.

The amount of glidant used in the composition may be in the range of 0.1 wt.-% to 10 wt.-%, more preferably 2 wt.-% to 7 wt.-%.

The process of preparing the coating composition of the present invention includes combining the constituents of the composition, including film-former, plasticizer, and optional components. The combination of these constituents may be achieved by several processes, such as heating, stirring, mixing and the like to obtain a homogenous dispersion. After initial mixing, crospovidone having an average particle size of less than 10 μm and other pharmaceutical acceptable excipients may be added to obtain the composition of the present invention. The coating composition of the present invention has good adhesion properties and in particular good film forming properties.

The coating may be applied by spray coating processes commonly used to coat orally ingestible substrates. The amount of coating applied will depend upon several factors, including the nature and functionality of the coating, the substrate to be coated and the apparatus employed to apply the coating. The coating compositions of the present invention may also be suitable for high volume batch process and continuous coating operations.

In a further aspect, the invention relates to a coated tablet comprising
(a) a tablet core, and
(b) a coating comprising an opacifying amount of crospovidone having an average particle size of less than 10 μm.

Generally, the opacifying amount is 25 wt.-% or more, preferably 32 wt.-% or more, or 40 wt.-% or more, in some cases 50 wt.-% or more, of the total weight of the coating.

Preferably, the surface coverage of the coated tablet with the crospovidone is 1 mg/cm² or more, preferably 2 mg/cm² or more, more preferred 3 mg/cm² or more. Generally, the surface coverage of the coated tablet with the crospovidone is 9 mg/cm² or less. The surface coverage is intended to mean the amount of coating [in mg as solids] applied per geometrical surface area of the tablet [in cm²] times the proportion of the crospovidone in the coating.

The coated tablet may comprise a coating composition comprising a colorant and/or a pigment.

The tablet core may include pharmaceuticals, nutraceuticals and dietary supplements as well as any other art-recognized orally ingestible core. Generally, the tablet core comprises at least one pharmaceutically active ingredient.

Suitable pharmaceutically active ingredient that may be comprised in the tablet core include, but are not limited to: adrenergic blocking agents; acetyl-cholinesterase inhibitors; analgesic or antipyretics; angiotensin modulators; anthelmintic agents; anti-anxiety agents; antibacterial agents; antibiotics; anticoagulants; anticonvulsant agents; antidepressants; antifungal agents; antihistamines; antimalarial agents; antimicrobial agents; antipsychotic agents; antiviral agents; blood glucose lowering drugs; calcium channel modulators; diuretics; erectile dysfunction agents; gastric acid secretion inhibitors; histamine H2-receptor antagonists; inhibitors of steroid Type II 5α-reductase; lipid regulating agents; selective HI-receptor antagonists; vasodilators; and vitamins.

The coating composition is applicable to tablet cores that comprise one or more acidic pharmaceutically active ingredients. Pharmaceutically active ingredients having a pka value of 1 to 8 are considered acidic pharmaceutically active ingredients. These acidic active pharmaceutical ingredients cause incompatibilities with coatings containing basic pigments, such as carbonates. Crospovidone having an average particle size of less than 10 μm is not subject to such incompatibilities.

The core may include tableting excipients. Tableting excipients include fillers such as lactose, cellulose powder, calcium diphosphate, microcrystalline cellulose, sugar alcohols, e.g., mannitol, sorbitol and starch, disintegrants such as starch (derivatives), croscarmellose, cross-linked polyvinylpyrrolidone (crospovidone), carboxymethyl cellulose (CMC); lubricants such as stearic acid, magnesium stearate; glidants such as silicon dioxide (Aerosil); or mixtures thereof.

In a further aspect, the invention relates to the use of crospovidone having an average particle size of less than 10 μm as opacifier and white pigment in a coating of a coated tablet, in particular in a film coating of a film coated tablet.

### EXAMPLES

Tablet cores were produced as follows: A powder mixture was obtained by mixing the ingredients indicated in table 1 below. The mixture was tableted on a tableting press (9 mm concave punch, compression force (upper): 9.4 kN). The mean tablet weight was adjusted to 322.9 mg. The mean surface of an individual tablet was 2.14 cm².

TABLE 1

| | Amount | Amount |
|---|---|---|
| | [%] | [mg] |
| Nimodipin | 9.52 | 30.00 |
| Kollitab DC 87L | 84.13 | 264.99 |
| Kolliphor SLS fine | 3.18 | 10.00 |
| Pruv | 3.18 | 10.00 |
| total | 100.00 | 315.00 |

The tablet cores were further processed in a perforated drum coater to obtain film tablets at an inlet-air temperature of 55° C., a bed temperature of 37° C., an inlet air quantity of 280 cm³/h, a spray rate of 15 g/min, an atomization air pressure of 1 bar and a pattern air pressure of 1 bar.

Coating formulations 1 to 7 according to table 2 below were investigated:

TABLE 2
Coating formulations 1 to 7.

| | | Kollidon | | | |
|---|---|---|---|---|---|
| trial | Kollicoat IR | CL-M | Talc | TiO₂ | CaCO₃ |
| # | [%] | [%] | [%] | [%] | [%] |
| 1 | 50 | 50 | 0 | 0 | 0 |
| 2* | 66 | 0 | 30.5 | 3.5 | 0 |
| 3 | 15 | 85 | 0 | 0 | 0 |
| 4 | 25 | 75 | 0 | 0 | 0 |
| 5* | 50 | 0 | 50 | 0 | 0 |
| 6* | 50 | 0 | 0 | 0 | 50 |
| 7 | 50 | 25 | 0 | 0 | 25 |
| 8* | 25 | 0 | 67.3 | 7.7 | 0 |

*Reference example

Coatings were applied as 20% dispersions. The tablets were coated at coating levels ranging from 1 to 10 (12) mg/cm².

Nimodipine is a yellow API, consistently tablets are yellow as well. Yellowness of film-coated tablets was evaluated by determining color values associated with the L*a*b color model, using a DataColor 400 spectrophotometer.

The graphs indicate the change in color from yellow to white with increasing coating levels. Samples 3 (85 wt.-% Kollidon CL-M) and 4 (75 wt.-% Kollidon CL-M) outperform the reference 2 (30.5 wt.-% talc and 3.5 wt.-% TiO₂). In sample 3, the maximal pigment load was exceeded and chalking effects and coating defects were observed. For less demanding purposes, lower concentrations of crospovidone having an average particle size of less than 10 μm may be adequate.

### Shielding Power Evaluation

In this example, the shielding power of the coating compositions is evaluated by coating blue mock tablets with varying amounts of the coating compositions.

A tablet core of 100 wt.-% Kollitab DC 87L was prepared as described above. Said tablet core was sub-coated using a coating formulation of 1 wt.-% FD&C Blue and 99 wt.-% Kollicoat IR. The sub-coating was applied as 20% dispersion. The tablet core was sub-coated until a weight gain of 5% was achieved to obtain blue sub-coated tablets.

Subsequently, the blue sub-coated tablets were top-coated using coating formulations 4 and 8* of table 2. The top-coatings were applied as 20% dispersions. The tablets were top-coated at coating levels ranging from 4 to 24 mg/cm² (coating composition 4) and 4 to 28 mg/cm² (coating composition 8*). Top-coating was performed using a GEA CosiGma Coater at a drum speed of 100 rpm, an inlet-air temperature of 70° C., an air knife temperature of 75° C., a spray rate of 110 g/min, an atomization air pressure of 1.5 bar and a pattern air pressure of 1.3 bar.

The resulting top-coated tablets were investigated regarding their ΔE values. ΔE represents the distance between two colors in the CIE-Lab system. It is calculated from the coordinates of two-color locations. With the ΔE value, differences between two colors (that are perceived by human eye), can be assigned to numbers:

$$Δ E = \sqrt{{( {L}_{0} - {L}_{1} )}^{2} + {( {a}_{0} - {a}_{1} )}^{2} + {( {b}_{0} - {b}_{1} )}^{2}}$$

An “ideal white” has a “L value” of 100, an “a value” of 0, and a “b value” of 0. An “ideal black” has a “L value” of 0, an “a value” of 0, and a “b value” of 0.

The resulting ΔE values are shown in table 3.

TABLE 3

| | coating composition 4 | coating composition 8* |
|---|---|---|
| coating level [mg/cm²] | ΔE (ideal white) | ΔE (ideal white) |
| 0 | 70.79 ± 0.37 | 70.79 ± 0.37 |
| 4 | 15.75 ± 2.05 | 30.90 ± 2.09 |
| 6 | 10.77 ± 0.40 | 22.44 ± 1.00 |
| 10 | 6.65 ± 0.35 | 13.51 ± 0.13 |
| 16 | 4.12 ± 0.10 | 9.63 ± 0.55 |
| 20 | 4.14 ± 0.06 | 8.01 ± 0.35 |
| 24 | 3.83 ± 0.15 | 6.59 ± 0.04 |
| 28 | n.d. ^{[1]} | 6.00 ± 0.18 |

^{[1]} not determined
*Reference example

The tablets top-coated with the inventive coating composition 4 have a smaller deviation from ideal white compared to the tablets top-coated with the reference coating composition 8* (at the same coating levels). The visual impression of the tablets top-coated with the inventive coating compositions are closer to an “ideal white” at lower coating levels compared to the tablets top-coated with the reference coating composition.

### Coatings Comprising a Colorant/Pigment in Addition Crospovidone

Coloration of a Kollicoat IR (film-former) based Kollidon CL-M coating was tested on Ludipress LCE tablets.

A SilentCrusher M (available from Heidolph) was used for dispersing of pigments.

Tablet cores with the ingredients indicated in table 4 below were produced as described above. The tablet cores had a tensile strength of 2.78 N/mm², and a height of 5.05 mm.

TABLE 4

| | Amount | Amount |
|---|---|---|
| | [%] | [mg] |
| Ludipress LCE | 74.0 | 243.46 |
| Caffeine | 15.5 | 51.00 |
| Kollidon CL-F | 5.0 | 16.45 |
| Kollidon VA64 fine | 5.0 | 16.45 |
| Mg-stearate | 0.5 | 1.64 |
| total | 100.00 | 329.00 |

Coating formulations 9 and 10 according to table 5 below were investigated.

TABLE 5
Coating formulations 9 and 10.

| | | Kollidon | Iron oxide red | |
|---|---|---|---|---|
| | Kollicoat IR | CL-M | (E172) | Water |
| # | [%] | [%] | [%] | [%] |
| 9 | 13.28 | 6.62 | 0.10 | 80.00 |
| 10 | 10.00 | 9.90 | 0.10 | 80.00 |

For coating preparation, Kollicoat IR was dissolved in 70% of the total amount of water. Then, Kollidon CL-M was added, and the mixture was stirred for 1 h. Iron oxide red (E172) was dispersed for 10 min at 10.000 rpm in the remaining water quantity (30% of the total amount. The resulting suspension was added to the Kollicoat IR/Kollidon CL-M suspension.

The coating suspensions of table 5 were applied to the tablet cores of table 4 until a weight gain of 4% was achieved. Samples were taken at 1%, 2%, 3% and 4% weight gain (1%=95 g suspension). Good coloration was achieved from 3% weight gain upwards.

Coating formulations 11 to 19 according to table 6 below were investigated.

TABLE 6
Coating formulations 11 to 19.

| | | Kollidon | | | |
|---|---|---|---|---|---|
| | Kollicoat IR | CL-M | | Colorant | Water |
| # | [%] | [%] | Colorant | [%] | [%] |
| 11 | 10.00 | 9.95 | Amaranth 85 | 0.05 | 80.00 |
| | | | (E123) | | |
| 12 | 10.00 | 9.95 | Azorubine 85 | 0.05 | 80.00 |
| | | | (E122) | | |
| 13 | 10.00 | 9.95 | Quinoline 70 | 0.05 | 80.00 |
| | | | (E104) | | |
| 14 | 10.00 | 9.95 | Cocheneal Red 80 | 0.05 | 80.00 |
| | | | (E124) | | |
| 15 | 10.00 | 9.95 | Erythrosine 88 | 0.05 | 80.00 |
| | | | (E127) | | |
| 16 | 10.00 | 9.95 | FD&C Blue No. 1 | 0.05 | 80.00 |
| | | | (E133) | | |
| 17 | 10.00 | 9.95 | Yellow Orange 85 | 0.05 | 80.00 |
| | | | (E110) | | |
| 18 | 10.00 | 9.95 | Indigotine 85 | 0.05 | 80.00 |
| | | | (E132) | | |
| 19 | 10.00 | 9.95 | Patent Blue 85 | 0.05 | 80.00 |
| | | | (E131) | | |

For coating preparation, Kollicoat IR was dissolved in water. Colorant and Kollidon CL-M were added, and the mixture was stirred for 1 h.

The coating suspensions of table 6 were applied to the tablet cores of table 4 until a weight gain of 4% was achieved (4%=380 g suspension). Good and intense coloration was achieved.

1.-14. (canceled)

15. A coating composition comprising an opacifying amount of 25 wt.-% or more, relative to the solids content of the coating composition, of crospovidone having a De Brouckere mean diameter D[4,3] of less than 10 μm, obtained from a particle size distribution determined by means of laser defractometry using a Malvern Mastersizer X at a compressed-air atomization of 2 bar.

16. The coating composition according to claim 15, wherein the opacifying amount is 32 wt.-% or more, or 40 wt.-% or more, relative to the solids content of the coating composition.

17. The coating composition according to claim 15, comprising a colorant and/or a pigment.

18. The coating composition according to claim 15, wherein the coating composition is free of titanium dioxide.

19. The coating composition according to claim 15, containing a polymeric film-former.

20. The coating composition according to claim 19, wherein the polymeric film-former is selected from polyvinylpyrrolidone (PVP), polyvinylpyrrolidone-vinyl acetate copolymer (copovidone), methylcellulose (MC), ethyl cellulose (EC), hydroxypropyl cellulose (HPC), hydroxypropyl methylcellulose (HPMC), sodium carboxymethyl cellulose (sodium CMC), polydextrose, starch, modified starch, polyvinyl alcohol (PVA), copolymers based on polyvinyl alcohol, polyvinyl acetate phthalate, cellulose acetate phthalate, cellulose acetate trimellitate, hydroxypropyl methylcellulose phthalate, methacrylate-based polymers, or combinations thereof.

21. A coated tablet comprising
(a) a tablet core, and
(b) a coating comprising an opacifying amount of 25 wt.-% or more, relative to the solids content of the coating composition, of crospovidone having a De Brouckere mean diameter D[4,3] of less than 10 μm, obtained from a particle size distribution determined by means of laser defractometry using a Malvern Mastersizer X at a compressed-air atomization of 2 bar.

22. The coated tablet according to claim 21, wherein the opacifying amount is 32 wt.-% or more, or 40 wt.-% or more of the total weight of the coating.

23. The coated tablet according to claim 21, wherein the surface coverage of the coated tablet with the crospovidone is 1 mg/cm² or more.

24. The coated tablet according to claim 21, wherein the coating composition comprises a colorant and/or a pigment.

25. The coated tablet according to claim 21, wherein the coating is free of titanium dioxide.

26. The coated tablet according to claim 25, wherein the coating is further free of talc.

27. The coated tablet according to claim 21, wherein the tablet core comprises at least one pharmaceutically active ingredient.

28. The use of crospovidone having a De Brouckere mean diameter D[4,3] of less than 10 μm, obtained from a particle size distribution determined by means of laser defractometry using a Malvern Mastersizer X at a compressed-air atomization of 2 bar, as opacifier and white pigment in a coating of a coated tablet.
